SEQUENCE LISTING (7 sequences)

SEQ ID NO: 1 - protein, 167 aa, Acinetobacter baumannii
ACTPETGFKTLDVPMAVGRVVVRPTDEVGKVLRKATFPISSNGSTYWCDSYGGYLEANLTQNYPISSIGNNVYSTNIPGIGIRLYREAENATNFSGYYPYRAATQRSRRYTLATGYFVVEIVKTETQTGSGTLVPGRYSTYNASGYSAVPWLTSTVYGNAITIASSS

SEQ ID NO: 2 - protein, 167 aa, Acinetobacter baumannii
YCTLSSGFTTVDISMAVGRVVVRPSDPVGKILRKATFPINPNGSTLRCTSYSDTITAALTQNYPLSPLGNSIYSTNIPGIGIRLYREAENATNFSGYYPYTRSLTPGTTYNLAQGYFVVEIVKTADQTGSGTLVPGLYSRYYVNGHMDRPFLTSTVYGNAITIASSS

SEQ ID NO: 3 - protein, 168 aa, Acinetobacter baumannii
NCTLSKGFTTVDIPMTIGTIVVRPTDPIGTVLQKNTFTISPNNSTATCNRASDQITAALPLNYPVSSIGNNVYATNIPGIGIRLYREAFDSTDFSGYYPYKRSLTPNTTYTLSPGYFVMEVIKTAATTGSGALVAGRYSTYYVTGQQNRPFLTTTVLSSSPILIASSS

SEQ ID NO: 4 - protein, 185 aa, Klebsiella pneumoniae
ASWSHPQFEKSGGGGGLVPRGSGSSCWQSNSAYEINMAMGRVVVSPDLPVGSVIATKTWTMPDNNTIYVTCDRNTTLKSDAKVVAAGLVQGANKVYSTAIPGIGLRFSRKGAISMIYPDSYTTTGSSFRLAGSTFTLDIIKTSTTTGSGTLASGPYTEYGPGFTILKTSLNADAITIVSPSCTIL

SEQ ID NO: 5 - protein, 338 aa, Acinetobacter baumannii
MKRILLTGFLFTAGLGVYGEANAACTPETGFKTLDVPMAVGRVVVRPTDEVGKVLRKATFPISSNGSTYWCDSYGGYLEANLTQNYPISSIGNNVYSTNIPGIGIRLYREAENATNFSGYYPYRAATQRSRRYTLATGYFVVEIVKTETQTGSGTLVPGRYSTYNASGYSAVPWLTSTVYGNAITIASSSCEIQGNINKVVQLPTVTKAGFKGVGSTQGEQTFDMNILCNGGINPTGYEEKNVISLSYDFTQDGTNSQVLANTAAASEKANGVGVQLLWNYQNKNQVIKKGDKLALGTLSSNQTLQYNVPMTARYYQTATNVTAGKVRAMATVTIEYD

SEQ ID NO: 6 - protein, 335 aa, Acinetobacter baumannii
MLLTGFLFTAGLGVYGEANAYCTLSSGFTTVDISMAVGRVVVRPSDPVGKILRKATFPINPNGSTLRCTSYSDTITAALTQNYPLSPLGNSIYSTNIPGIGIRLYREAENATNFSGYYPYTRSLTPGTTYNLAQGYFVVEIVKTADQTGSGTLVPGLYSRYYVNGHMDRPFLTSTVYGNAITIASSSCEIQGNINKVVQLPTVTKAGFKGVGSTQGDQTFDMNILCNGGINPTGYEEKNLISLTYDFTQDGTNNQVLANTAPTSEKANGVGVQLLWNYQNKNQVIKKGDKLALGTLSSNQTIQYNVPMTARYYQTATNVTAGKVRAMATVTIEYD

SEQ ID NO: 7 - protein, 339 aa, Acinetobacter baumannii
MKKMLIKSGLLITSFFIYNQTYANCTLSKGFTTVDIPMTIGTIVVRPTDPIGTVLQKNTFTISPNNSTATCNRASDQITAALPLNYPVSSIGNNVYATNIPGIGIRLYREAFDSTDFSGYYPYKRSLTPNTTYTLSPGYFVMEVIKTAATTGSGALVAGRYSTYYVTGQQNRPFLTTTVLSSSPILIASSSCEIQNGIDTVVQLPTVMKSGFRAIGSTQGEKNFNLSILCNGGENNSGVPTSNTLSLSFDYISDTSNSQVINNSAADSTKANGVGVELLWNMNGANNPIRKGNKLDIGTVSSNQTVQYDISLTARYYQTASNVTAGEVKANATVTIQYD

(19) United States
(12) Patent Application Publication
(10) Pub. No.: US 2026/0191944 A1
(43) Pub. Date: Jul. 9, 2026

(54) ACINETOBACTER VACCINE AND METHOD OF USE THEREOF

(71) Applicant: Washington University, St. Louis, MO (US)

(72) Inventors: Scott HULTGREN, St. Louis, MO (US); Morgan TIMM, St. Louis, MO (US); Kevin TAMADONFAR, St. Louis, MO (US); Ali ELLEBEDY, St. Louis, MO (US); Jerome PINKNER, St. Louis, MO (US); Mario FELDMAN, St. Louis, MO (US); Karen DODSON, St. Louis, MO (US)

(73) Assignee: Washington University, St. Louis, MO (US)

(21) Appl. No.: 19/133,520

(22) PCT Filed: Dec. 5, 2023
(86) PCT No.: PCT/US2023/082426

§ 371 Date: May 28, 2025

Related U.S. Application Data
(60) Provisional application No. 63/430,087, filed on Dec. 5, 2022.

(51) Int. Cl. A61K 39/104; A61K 39/00; A61P 31/04; C07K 14/21

(52) U.S. Cl. CPC A61K 39/1045; A61P 31/04; C07K 14/212; A61K 2039/55566; A61K 2039/575

(57) ABSTRACT

The present disclosure provides for a vaccine comprising an Acinetobacter adhesin protein or fragment thereof and a pharmaceutically acceptable carrier or adjuvant. The vaccine may be used for treating or preventing a urinary tract infection (UTI) in a subject in need thereof, particularly a catheter-associated UTI (CAUTI). In some embodiments, the Acinetobacter adhesin protein or fragment thereof is an isolated Acinetobacter chaperone-usher pathway (CUP) adhesin protein or immunogenic fragment thereof.

### CROSS-REFERENCE TO RELATED APPLICATIONS

This application claims priority from U.S. Provisional Application Ser. No. 63/430,087 filed on 5 Dec. 2022, which is incorporated herein by reference in its entirety.

### STATEMENT REGARDING FEDERALLY SPONSORED RESEARCH OR DEVELOPMENT

This invention was made with government support under A1157797 and A1048689 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “020120-WO_Sequence_Listing.xml” created on 1 Dec. 2023; 8,574 bytes). The subject matter of the Sequence Listing is incorporated herein by reference in its entirety.

### FIELD

The present disclosure generally relates to vaccines comprising an Acinetobacter adhesin protein.

### SUMMARY

Among the various aspects of the present disclosure is the provision of Acinetobacter vaccines and methods of use thereof.

In one aspect of the present disclosure, a vaccine is provided. The vaccine comprises at least one isolated Acinetobacter chaperone-usher pathway (CUP) adhesin protein or immunogenic fragment thereof; and a pharmaceutically acceptable carrier or adjuvant.

In some embodiments, the at least one isolated Acinetobacter CUP adhesin protein or immunogenic fragment thereof is derived from Acinetobacter baumannii.

In some embodiments, the at least one isolated Acinetobacter CUP adhesin protein or immunogenic fragment thereof is Abp2D, Abp1D, or a combination thereof. In certain embodiments, the at least one isolated Acinetobacter CUP adhesin protein or immunogenic fragment thereof is Abp2D; and the vaccine does not comprise Abp1 D.

In some embodiments, the at least one isolated Acinetobacter CUP adhesin protein or immunogenic fragment thereof is selected from the group consisting of a receptor binding domain (RBD) of Abp2D, an RBD of Abp1 D, and a combination thereof. In certain embodiments, the at least one isolated Acinetobacter CUP adhesin protein or immunogenic fragment thereof is an RBD of Abp2D; and the vaccine does not comprise an RBD of Abp1 D.

In some embodiments, the at least one isolated Acinetobacter CUP adhesin protein or immunogenic fragment thereof comprises a polypeptide sequence selected from the group consisting of SEQ ID NO: 1, SEQ ID NO: 2, SEQ ID NO: 3, SEQ ID NO: 5, SEQ ID NO: 6, SEQ ID NO: 7, and combinations thereof. In some embodiments, the at least one isolated Acinetobacter CUP adhesin protein or immunogenic fragment thereof comprises a polypeptide sequence having at least about 70% identity to SEQ ID NO: 1, SEQ ID NO: 2, SEQ ID NO: 3, SEQ ID NO: 5, SEQ ID NO: 6, or SEQ ID NO: 7.

In some embodiments, the pharmaceutically acceptable carrier or adjuvant is selected from the group consisting of a cream, emulsion, gel, liposome, nanoparticle, or ointment. In certain embodiments, the pharmaceutically acceptable carrier or adjuvant is squalene.

In another aspect of the present disclosure, a method of treating or preventing a urinary tract infection (UTI) in a subject in need thereof is provided. The method comprises administering to the subject a vaccine comprising at least one isolated Acinetobacter chaperone-usher pathway (CUP) adhesin protein or immunogenic fragment thereof and a pharmaceutically acceptable carrier or adjuvant.

In some embodiments, the UTI is a catheter-associated UTI (CAUTI). In certain embodiments, the subject is a chronically catheterized subject or has recurrent CAUTI.

In some embodiments, wherein the at least one isolated Acinetobacter CUP adhesin protein or immunogenic fragment thereof is derived from Acinetobacter baumannii.

In some embodiments, the at least one isolated Acinetobacter CUP adhesin protein or immunogenic fragment thereof is Abp2D, Abp1D, or a combination thereof. In certain embodiments, the at least one isolated Acinetobacter CUP adhesin protein or immunogenic fragment thereof is Abp2D; and the vaccine does not comprise Abp1 D.

In some embodiments, the at least one isolated Acinetobacter CUP adhesin protein or immunogenic fragment thereof is selected from the group consisting of a receptor binding domain (RBD) of Abp2D, an RBD of Abp1 D, and a combination thereof.

In some embodiments, the at least one isolated Acinetobacter CUP adhesin protein or immunogenic fragment thereof is an RBD of Abp2D; and the vaccine does not comprise an RBD of Abp1 D. In certain embodiments, the at least one isolated Acinetobacter CUP adhesin protein or immunogenic fragment thereof comprises a polypeptide sequence selected from the group consisting of SEQ ID NO: 1, SEQ ID NO: 2, SEQ ID NO: 3, SEQ ID NO: 5, SEQ ID NO: 6, SEQ ID NO: 7, and combinations thereof. In other embodiments, the at least one isolated Acinetobacter CUP adhesin protein or immunogenic fragment thereof comprises a polypeptide sequence having at least about 70% identity to SEQ ID NO: 1, SEQ ID NO: 2, SEQ ID NO: 3, SEQ ID NO: 5, SEQ ID NO: 6, or SEQ ID NO: 7.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1C is an exemplary embodiment showing immunity from prior A. baumannii infection in accordance with the present disclosure. FIG. 1A is a schematic showing 6-7 week old C57/Bl6 mice were catheterized and infected with A. baumannii strain ACICU or mock-infected with PBS. Urine was collected to monitor infection status. Mice received 10 days of oral apramycin (1 mg/L) at Week 5 to clear the infection. At week 8, mice were catheterized and challenged with A. baumannii strain ACICU and sacrificed 24 hours post infection. FIG. 1B includes heat maps showing bladder and kidney homogenates and serum from Weeks 3, 5, and 8 were assayed for Abp1 D_{RBD}- and Abp2D_{RBD}-specific IgG by ELISA. Heatmaps were generated by calculating the area under the curve for each serum/tissue sample. FIG. 1C includes dot plots showing bladder, catheter, and kidney titers were enumerated. Dashed lines indicate limit of detection. Solid lines indicate geometric mean+/−geometric SD. Data shown are pooled from two independent experiments, n=17 (mock) and n=18 (ACICU). Mann-Whitney U-test, ***P≤0.0005, **P≤0.005.

FIG. 2A-FIG. 2E is an exemplary embodiment showing immunization with Abp1 D and Abp2D provides protection from CAUTI in accordance with the present disclosure. FIG. 2A is a schematic showing 6-7 week old C57/Bl6 mice received 3 adjuvanted doses of 50 ug Abp1 D_{RBD} and 50 ug Abp2D_{RBD} or buffer alone (mock). Serum was collected at weeks 4 and 8 prior to immunizations, and at week 12 following sacrifice. Four weeks after the third dose, mice were catheterized and challenged with A. baumannii strain ACICU. Mice were sacrificed 24 hours after infection. FIG. 2B includes heat maps showing bladder and kidney homogenates and serum from Week 4, Week 8, and the day of sacrifice were assayed for Abp1 D_{RBD} and Abp2D_{RBD}-specific IgG by ELISA. Heatmaps were generated by calculating area under the curve for each serum/tissue sample. FIG. 2C includes dot plots showing bacterial titers were enumerated from bladders, catheters, and kidneys. Dashed lines indicate limit of detection. Solid lines indicate geometric mean+/−geometric SD. Data shown are pooled from two independent experiments, n=19 per 593 group. FIG. 2D is a dot plot showing normalized reduction in bacterial titers in the bladders of convalescent and Abp1D_{RBD}/Abp2D_{RBD} immunized mice. Bars indicate mean+/−SD. FIG. 2E is a bar graph showing ELISA AUCs of Abp1 D_{RBD} and Abp2D_{RBD}-specific IgG in serum from convalescent vs. Abp1 D_{RBD}/Abp2D_{RBD} immunized mice. Bars indicate mean+/−SD. Mann-Whitney U597 test, ***P≤0.0005, **P≤0.005, *P≤0.05.

FIG. 3A-FIG. 3C is an exemplary embodiment showing immunity to Abp2D, not Abp1D, drives protection from CAUTI in accordance with the present disclosure. FIG. 3A is a schematic showing 6-7 week old C57/Bl6 mice received 3 adjuvanted doses of 50 ug Abp1 D_{RBD}, Abp2D_{RBD}, or buffer alone (mock). Serum was collected at weeks 4 and 8 prior to immunizations, and at week 12 following sacrifice. Four weeks after the third dose, mice were catheterized and challenged with A. baumannii strain ACICU and sacrificed 24 hours post-infection. FIG. 3B includes heat maps showing bladder and kidney homogenates and serum from week 4, week 8, and the day of sacrifice were assayed for Abp1D_{RBD} and Abp2D_{RBD}-specific IgG by ELISA. Heatmaps were generated by calculating area under the curve for each serum/tissue sample. FIG. 3C includes dot plots showing bacterial titers were enumerated from bladders, catheters, and kidneys. Dashed lines indicate limit of detection. Solid lines indicate geometric mean+/−geometric SD. Data shown are pooled from two independent experiments, n=20 (Mock), n=17 (Abp1D_{RBD}), n=18 (Abp2D_{RBD}). Kruskal-Wallace test with multiple comparisons correction, ****P≤0.0001, **P≤0.005.

FIG. 4A-FIG. 4G is an exemplary embodiment showing vaccination with Abp2D generates antigen-specific memory B cells and bone marrow plasma cells in accordance with the present disclosure. FIG. 4A is a schematic showing 6-7 week old C57/Bl6 mice received 3 adjuvanted doses of 50 ug Abp2D_{RBD} or buffer (mock) and were sacrificed 4 weeks after the 3^{rd} dose. FIG. 4B includes graph showing splenic memory B cells (Live/CD4− CD19+/lgDlo/GL7− CD38+/IgG1+) were stained with Abp2D_{RBD}-biotin and detected with SA-APC-Fire750 via flow cytometry. FIG. 4C is a dot plot showing quantification of Abp2D_{RBD}⁺ splenic memory B cells as % of IgG1+ memory B cells. FIG. 4D is a dot plot showing total Abp2D_{RBD}⁺ memory B cells per spleen. Line indicates mean+/−SD. FIG. 4E includes images showing bone marrow was assayed for antigen-specific bone marrow plasma cells via ELISpot. Representative wells from mock-immunized animals and Abp2D_{RBD}-immunized animals are shown. FIG. 4F is a dot plot showing quantification of Abp2D_{RBD}-specific bone marrow plasma cells in Abp2D_{RBD}-immunized animals (n=10). Dotted line indicates the limit of detection (3 cells per 1e6 bone marrow cells). Solid line indicates mean+/−SD. FIG. 4G includes heat maps showing bladder and kidney homogenates and serum from week 4, week 8, and the day of sacrifice were assayed for Abp1 D_{RBD}- and Abp2D_{RBD}-specific IgG by ELISA. Heatmaps were generated by calculating area under the curve for each serum/tissue sample.

FIG. 5A-FIG. 5C is an exemplary embodiment showing passive immunization with serum from immunized mice protects naïve mice from CAUTI in accordance with the present disclosure. FIG. 5A is a schematic showing 6-8 week old C56/Bl6 mice received two 100 ul doses of pooled serum at 3 hours prior and 12 hours after catheterization and infection with A. baumannii strain ACICU. Serum was pooled from i) mock immunized and mock infected animals, ii) convalescent animals, or animals immunized with iii) Abp1 D_{RBD}+Abp2D_{RBD}, iv) Abp1 D_{RBD} alone, or v) Abp2D_{RBD} alone. FIG. 5B includes dot plots showing mice were sacrificed 24 hours post-infection and bacterial titers enumerated from bladders, catheters, and kidneys. Dashed lines indicate limit of detection. Solid lines indicate geometric mean+/−geometric SD. Data shown are pooled from three independent experiments, n=12 (Mock), n=14 for all other groups. FIG. 5C includes heat maps showing serum, bladder and kidney homogenates from infected mice were assayed for Abp1 D_{RBD}- and Abp2D_{RBD}-specific IgG by ELISA. The serum pools used for immunizations were also tested and are shown on the left of each heatmap. Heatmaps were generated by calculating the area under the curve. Mann-Whitney U-test, *P≤0.05.

FIG. 6 includes graphs showing flow cytometry gating strategy for Abp2D+ splenic memory B cells in accordance with the present disclosure. Splenocytes were stained and gated on lymphocytes/single cells/live/CD4− CD19+/lgDlo/GL7− CD38+/IgG1+/Abp2D_{RBD}+.

FIG. 7 is an alignment showing sequences of Acinetobacter baumannii Abp2D and Abp1D receptor binding domains (RBD) (SEQ ID NOs: 1-3) from two strains aligned to Klebsiella pneumoniae adhesin, MrkD (SEQ ID NO: 4) in accordance with the present disclosure. The fibrinogen binding pocket is underlined. ACICU is a clinical meningitis isolate. UPAB1 is a clinical urinary tract isolate. These proteins were used as immunogens in vaccination studies.

FIG. 8A-FIG. 8B show crystal structures of Abp2D (ACICU) (FIG. 8A) and Abp1D (UPAB1) (FIG. 8B) RBDs in accordance with the present disclosure.

FIG. 9A-FIG. 9B include graphs showing WT Abp2D (FIG. 9A) and Abp1 D and (FIG. 9B) bind to fibrinogen in an ELISA-based assay. Mutation of residues within the binding pocket (R86E among others; data in red) reduces adhesion.

### DETAILED DESCRIPTION

The present disclosure is based, at least in part, on the discovery of a protein subunit vaccine that may be used for the treatment of catheter-associated urinary tract infections (CAUTIs) caused by Acinetobacter baumannii.

Acinetobacter baumannii is classified as a pathogen of urgent concern by the CDC due to its high level of antibiotic resistance. It causes infections in numerous body systems including the urinary tract and respiratory system. Given the increasing prevalence of antibiotic resistance and the difficulty of treating highly resistant A. baumannii infections, there is an urgent need for antibiotic-sparing therapeutics, such as the vaccine described herein.

Catheter-associated urinary tract infections (CAUTIs) contribute greatly to the burden of healthcare associated infections. Acinetobacter baumannii is a Gram-negative bacterium with high levels of antibiotic resistance that is of increasing concern as a CAUTI pathogen. A. baumannii expresses fibrinogen-binding adhesins (Abp1 D and Abp2D) that mediate colonization and biofilm formation on catheters, which become coated with fibrinogen upon insertion. Described herein is a protein subunit vaccine against Abp1D_{RBD} and Abp2D_{RBD} that significantly reduced bladder bacterial titers in a mouse model of CAUTI (see e.g., Example 1). Immunity to Abp2D_{RBD} alone was found to be sufficient for protection. Mechanistically, the B cell response to Abp2D_{RBD} vaccination was defined and it was demonstrated that immunity was transferrable to naive mice through passive immunization with Abp2D_{RBD}-immune sera. This work represents a novel strategy in the prevention of A. baumannii CAUTI and has an important role to play in the global fight against antimicrobial resistance.

### Adhesin Protein

The present disclosure provides for a vaccine comprising at least one isolated Acinetobacter chaperone-usher pathway (CUP) adhesin protein or an immunogenic fragment thereof.

Adhesins are specialized cell-surface proteins produced by bacteria that mediate attachment to host cells or surfaces. As described herein, A. baumannii chaperone-usher pathway (CUP) adhesins are critical in catheter colonization and thus represent promising drug targets. A. baumannii have evolved two CUP pili, Abp1 and Abp2, tipped with fibrinogen binding adhesins Abp1D and Abp2D respectively. The majority of published A. baumannii genomes encode one or both of these pilus operons. Both of these adhesins have been shown to bind fibrinogen and to be critical in a mouse model of CAUTI.

In some embodiments, the vaccine comprises the adhesin protein Abp2D or an immunogenic fragment thereof. For example, the immunogenic fragment thereof can be the receptor binding domain (RBD) of Abp2D. For example, the adhesin protein or immunogenic fragment thereof may comprise the polypeptide sequence of SEQ ID NO: 1, SEQ ID NO: 2, SEQ ID NO: 5, or SEQ ID NO: 6 as shown below:

Abp2D from Acinetobacter baumannii
UPAB1 strain (SEQ ID NO: 5) with
RBD (SEQ ID NO: 1) depicted in bold text:
MKRILLTGFL FTAGLGVYGE
ANAACTPETG FKTLDVPMAV
GRVVVRPTDE VGKVLRKATF
PISSNGSTYW CDSYGGYLEA
NLTQNYPISS IGNNVYSTNI
PGIGIRLYRE AENATNFSGY
YPYRAATQRS RRYTLATGYF
VVEIVKTETQ TGSGTLVPGR
YSTYNASGYS AVPWLTSTVY
GNAITIASSS CEIQGNINKV
VQLPTVTKAG FKGVGSTQGE
QTFDMNILCN GGINPTGYEE
KNVISLSYDF TQDGTNSQVL
ANTAAASEKA NGVGVQLLWN
YQNKNQVIKK GDKLALGTLS
SNQTLQYNVP MTARYYQTAT
NVTAGKVRAM ATVTIEYD
Abp2D from Acinetobacter baumannii
ACICU strain (SEQ ID NO: 6) with
RBD (SEQ ID NO: 2) depicted in bold text:
MLLTGFLFTA GLGVYGEANA
YCTLSSGFTT VDISMAVGRV
VVRPSDPVGK ILRKATFPIN
PNGSTLRCTS YSDTITAALT
QNYPLSPLGN SIYSTNIPGI
GIRLYREAEN ATNFSGYYPY
TRSLTPGTTY NLAQGYFVVE
IVKTADQTGS GTLVPGLYSR
YYVNGHMDRP FLTSTVYGNA
ITIASSSCEI QGNINKVVQL
PTVTKAGFKG VGSTQGDQTF
DMNILCNGGI NPTGYEEKNL
ISLTYDFTQD GTNNQVLANT
APTSEKANGV GVQLLWNYQN
KNQVIKKGDK LALGTLSSNQ
TIQYNVPMTA RYYQTATNVT
AGKVRAMATV TIEYD

In some embodiments, the vaccine comprises the adhesin protein Abp1 D or an immunogenic fragment thereof. For example, the immunogenic fragment thereof can be the receptor binding domain (RBD) of Abp1D. For example, the adhesin protein or immunogenic fragment thereof may comprise the polypeptide sequence of SEQ ID NO: 3 or SEQ ID NO: 7 as shown below:

Abp1D from Acinetobacter baumannii
UPAB1 strain (SEQ ID NO: 7) with
RBD (SEQ ID NO: 3) depicted in bold text:
MKKMLIKSGL LITSFFIYNQ
TYANCTLSKG FTTVDIPMTI
GTIVVRPTDP IGTVLQKNTF
TISPNNSTAT CNRASDQITA
ALPLNYPVSS IGNNVYATNI
PGIGIRLYRE AFDSTDFSGY
YPYKRSLTPN TTYTLSPGYF
VMEVIKTAAT TGSGALVAGR
YSTYYVTGQQ NRPFLTTTVL
SSSPILIASS SCEIQNGIDT
WVQLPTVMKS GFRAIGSTQG
EKNFNLSILC NGGENNSGVP
TSNTLSLSFD YISDTSNSQV
INNSAADSTK ANGVGVELLW
NMNGANNPIR KGNKLDIGTV
SSNQTVQYDI SLTARYYQTA
SNVTAGEVKA NATVTIQYD

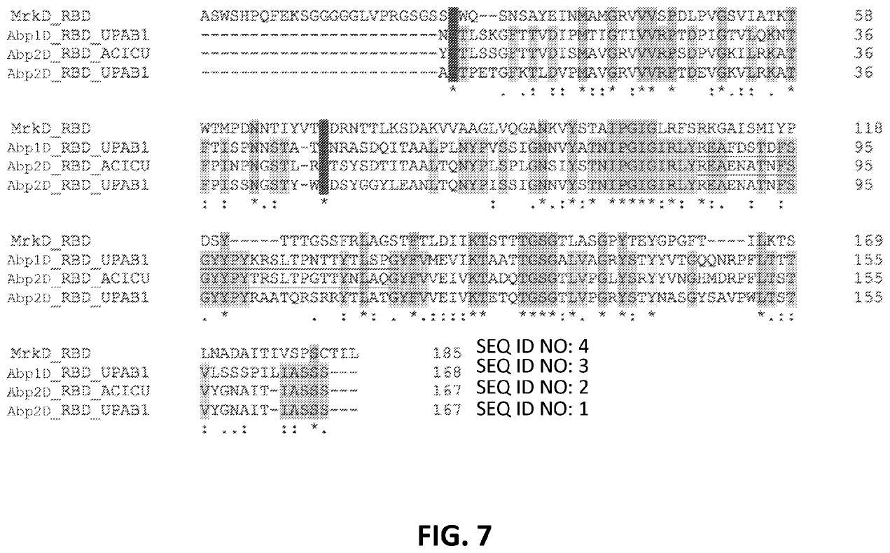

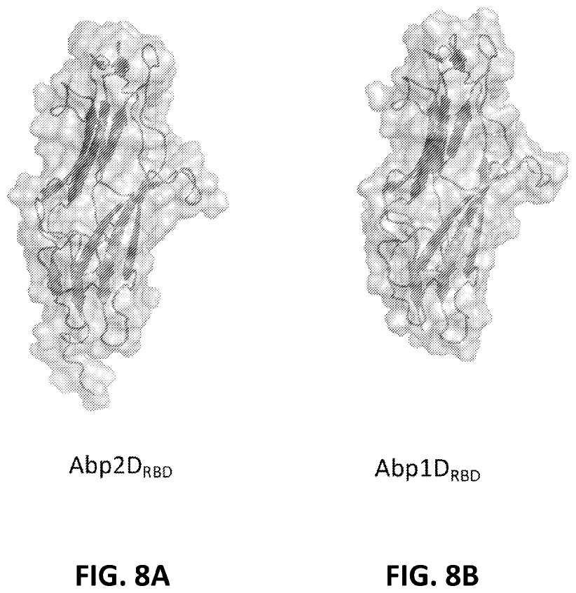

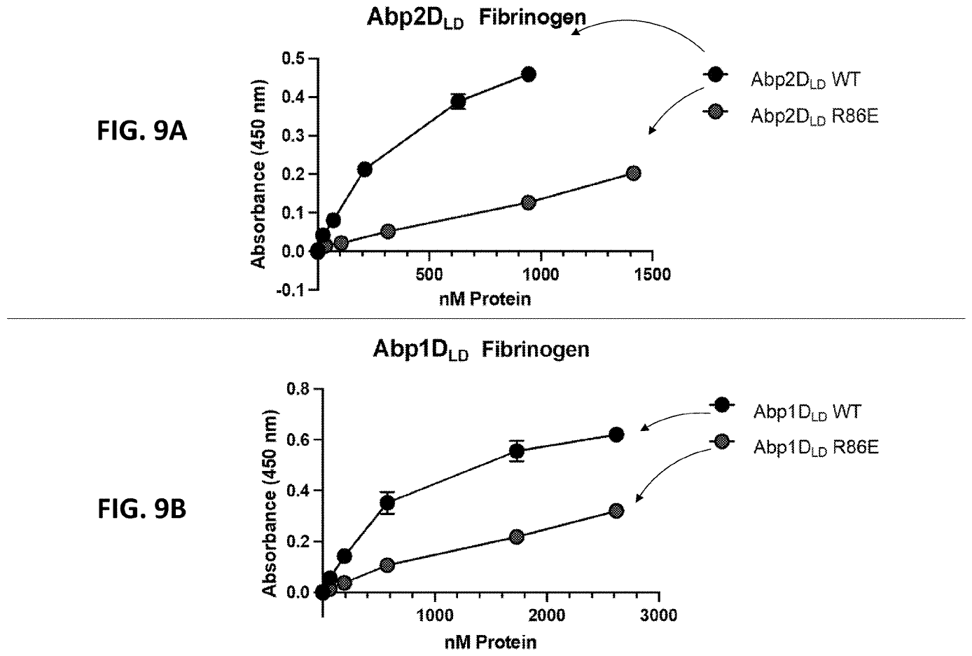

In some embodiments, the vaccine comprises a polypeptide sequence having at least about 70% identity to SEQ ID NO: 1, SEQ ID NO: 2, SEQ ID NO: 3, SEQ ID NO: 5, SEQ ID NO: 6, or SEQ ID NO: 7. There exists naturally occurring variation in the Abp2D or Abp1 D sequences of different Acinetobacter species or even between isolates of the same species, and it is expected that this naturally occurring variation does not significantly impact the immunogenicity of Abp2D or Abp1 D or fragments thereof. For example, a BLAST sequence alignment of the RBD regions of A. baumannii isolates UPAB1 and ACICU (as shown in FIG. 7) results in a sequence identity of about 70%.

As described herein, the vaccine of the present disclosure comprises an isolated CUP adhesin protein or immunogenic fragment thereof from a species of the genus Acinetobacter. Acinetobacter is a genus of gram-negative bacteria associated with nosocomial infections such as bacteremia, urinary tract infection (UTI), meningitis, endocarditis, and skin infection. For example, the Acinetobacter species can be A. baumannii, A. seifertii, A. nosocomialis, A. pittii, A. calcoaceticus, A. lactucae, A. oleivorans, A. soli, A. higginsii, A. johnsonii, A. junii, or A venetianus.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product—consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes fora different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log₁₀[Na⁺])+0.41 (fraction G/C content)−0.63 (% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing a urinary tract infection (UTI) in a subject in need of administration of a therapeutically effective amount of vaccine comprising an Acinetobacter adhesin protein or fragment thereof, so as to reduce bacterial titers, prevent infection, or produce a robust antigen-specific IgG response.

Methods described herein are generally performed on a subject in need thereof. A subject in need of the therapeutic methods described herein can be a subject having, diagnosed with, suspected of having, or at risk for developing a UTI. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a vaccine comprising an Acinetobacter adhesin protein or fragment thereof is, for example, an amount that would cause the desired therapeutic effect in a subject while minimizing undesired side effects. In various embodiments, an effective amount of vaccine comprising an Acinetobacter adhesin protein or fragment thereof described herein can substantially inhibit Acinetobacter adhesion, slow the progress of an Acinetobacter infection, or limit Acinetobacter growth or viability

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a vaccine comprising an Acinetobacter adhesin protein or fragment thereof can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce bacterial titers, prevent infection, or produce a robust antigen-specific IgG response.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a vaccine comprising an Acinetobacter adhesin protein or fragment thereof can occur as a single event or over a time course of treatment. For example, a vaccine comprising an Acinetobacter adhesin protein or fragment thereof can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for UTI.

A vaccine comprising an Acinetobacter adhesin protein or fragment thereof can be administered simultaneously or sequentially with another agent, such as an antibiotic, an anti-inflammatory, or another agent. For example, a vaccine comprising an Acinetobacter adhesin protein or fragment thereof can be administered simultaneously with another agent, such as an antibiotic or an anti-inflammatory. Simultaneous administration can occur through administration of separate compositions, each containing one or more of a vaccine comprising an Acinetobacter adhesin protein or fragment thereof, an antibiotic, an anti-inflammatory, or another agent. Simultaneous administration can occur through administration of one composition containing two or more of a vaccine comprising an Acinetobacter adhesin protein or fragment thereof, an antibiotic, an anti-inflammatory, or another agent. A vaccine comprising an Acinetobacter adhesin protein or fragment thereof can be administered sequentially with an antibiotic, an anti-inflammatory, or another agent. For example, a vaccine comprising an Acinetobacter adhesin protein or fragment thereof can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the vaccine comprising an Acinetobacter adhesin protein or fragment thereof may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a vaccine comprising an Acinetobacter adhesin protein or fragment thereof such as a compound as described herein may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to an Acinetobacter adhesin protein or fragment thereof, a pharmaceutically acceptable carrier or adjuvant, or a delivery or injection device. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Vaccination with Acinetobacter baumannii Adhesin Abp2D Provides Protection Against Catheter-Associated Urinary Tract Infection

This Example describes a protein subunit vaccine for the treatment of catheter-associated urinary tract infections caused by Acinetobacter baumannii.

### Introduction

The U.S. Centers for Disease Control and Prevention (CDC) estimates that 1 out of every 31 hospitalized patients in the U.S. will acquire at least one healthcare-associated infection (HAI) over the course of their care. HAIs are problematic because they lead to an increased burden of morbidity and mortality for patients, cost the US healthcare system an estimated $35.7 billion per year, and drive antibiotic overuse that contributes to the development of antimicrobial resistance. One of the most common types of HAIs are catheter-associated urinary tract infections (CAUTIs). The risk of CAUTI increases by 3-7% with each subsequent day of catheterization and approaches 100% in patients catheterized for more than 30 days. More than 30 million Foley (urinary) catheters are used annually in the United States. Because catheter use is so ubiquitous, CAUTIs make up nearly 40% of all HAIs in the US each year. Unfortunately, despite concerted infection mitigation efforts by public health agencies and healthcare facilities, the rate of CAUTIs continues to rise and increased by 5% from 2019-2021 in the United States.

Acinetobacter baumannii is a Gram-negative bacterium implicated in multiple types of HAIs. While it is best known for causing ventilator-associated pneumonia, it is increasingly recognized as an important CAUTI pathogen. Several single-center studies have identified A. baumannii as a leading cause of CAUTI in their facilities. Indeed, recent studies have demonstrated that 17% of published A. baumanni isolates originated in the urinary tract and up to 2% of the healthy population may exhibit A. baumannii asymptomatic bacteriuria. At the same time, the level of antimicrobial resistance identified in A. baumannii isolates is on the rise, with most isolates resistant to at least one antibiotic class and many isolates displaying multi-drug resistance. As a result, the CDC and World Health Organization have classified carbapenem-resistant A. baumannii as an “urgent threat,” which is the highest threat level. Together, the prevalence of A. baumannii coupled with its multi-drug resistance profile have emphasized the critical need for antibiotic-sparing therapeutics for A. baumannii CAUTI.

Many Gram-negative bacteria produce hair-like proteinaceous fibers called pili, tipped by specialized adhesins that recognize receptors with stereochemical specificity to determine host and tissue tropisms. The class of pili most implicated in host-pathogen interactions are the chaperone-usher pathway (CUP) pili. CUP adhesins are two-domain proteins with an amino terminal receptor binding domain (RBD) and a pilin domain that links the adhesin to the pilus rod. Importantly, new therapies that neutralize the function of the receptor binding domain of CUP pilus adhesins in a variety of pathogens have been successful in preclinical models and early human clinical trials.

Recent studies of A. baumannii CAUTI pathogenesis have revealed that A. baumannii CUP adhesins are critical in catheter colonization and thus may represent promising drug targets (see e.g., Di Venanzio et al. (2019) Nat. Commun. 10(2763) and Tamadonfar et al. (2023) Proc. Natl. Acad. Sci. U.S.A. 120, e2212694120). When a urinary catheter is inserted into the bladder, it induces inflammation and leads to deposition of host proteins, such as fibrinogen, onto the surface of the implant. A. baumannii have evolved two CUP pili, Abp1 and Abp2, tipped with fibrinogen binding adhesins Abp1 D and Abp2D respectively. The majority of published A. baumannii genomes encode one or both of these pilus operons. Both of these adhesins have been shown to bind fibrinogen and to be critical in a mouse model of CAUTI. Therefore, it was hypothesized herein that targeting the Abp1 D/Abp2D adhesins with an adhesin-based vaccine, comprised of their receptor binding domains Abp1 D_{RBD} and Abp2D_{RBD}, would prevent A. baumannii CAUTI pathogenesis.

Herein is described evidence that vaccination with recombinant Abp2D_{RBD} provides protection from A. baumannii CAUTI in a mouse model. Immunity conferred by a previous A. baumannii infection was used to establish a baseline for vaccine performance. Demonstrated herein is that a vaccine formulation containing both adhesins provided protection from A. baumannii CAUTI that was superior to natural infection. Further, immunity to Abp2D_{RBD} alone was shown to be sufficient for protection. Demonstrated herein is that the Abp2D_{RBD} vaccine generates robust memory B cell and bone marrow plasma cell responses, and that antibody-mediated protection is transferable to naive mice via passive immunization. This work provides proof-of-concept that an adhesin-based vaccine may be a promising strategy for multidrug-resistant A. baumannii CAUTI and could directly contribute to the arsenal of antibiotic-sparing therapeutics needed to meet the urgent threat of antibiotic-resistant A. baumannii.

### Results

### Natural Infection Provides Protection Against Subsequent A. Baumannii CAUTI Infection Despite Lack of Adhesin-Specific IgG Response.

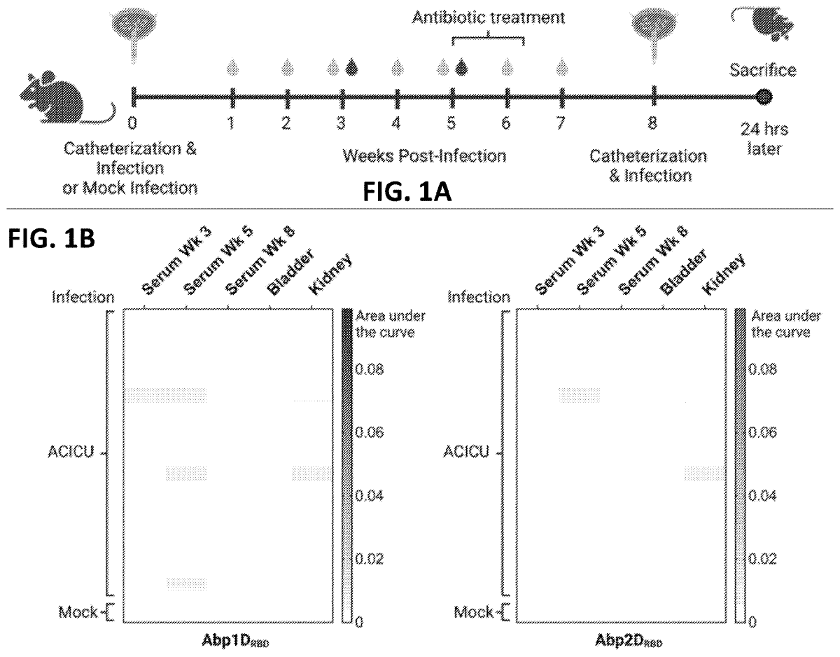

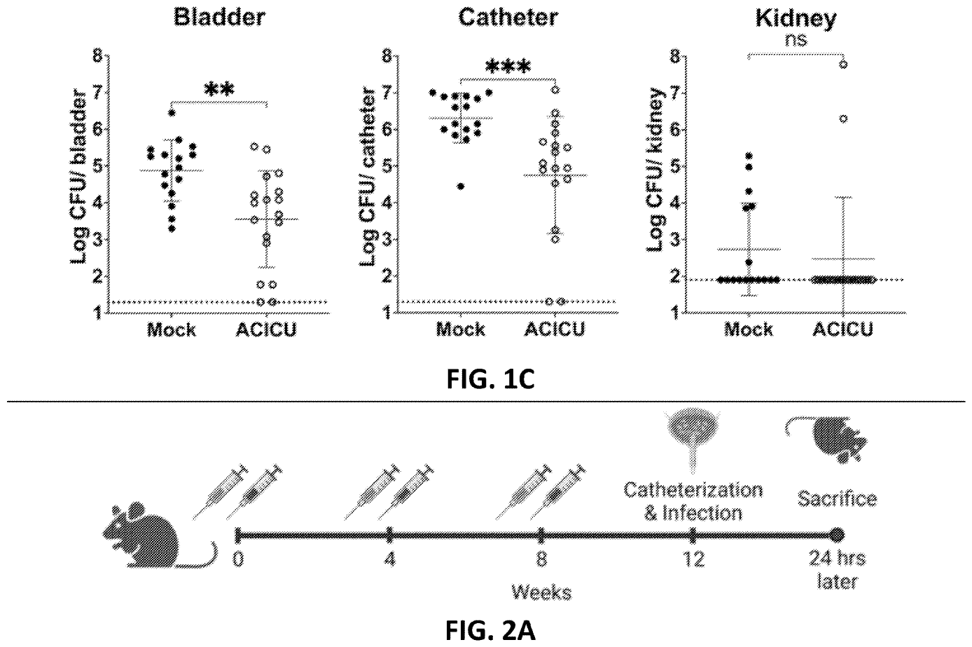

It was previously shown that adhesins Abp1 D and Abp2D are critical virulence factors for A. baumannii CAUTI pathogenesis (see e.g., Tamadonfar et al. (2023) Proc. Natl. Acad. Sci. U.S.A. 120, e2212694120). However, prior studies did not examine the immune response to Abp1 D/Abp2D during infection, nor did they consider whether a history of infection would provide any degree of protection from a subsequent challenge infection. To investigate this question, C57BL/6 mice were catheterized, infected with A. baumannii strain ACICU or mock-infected with PBS, and then treated with apramycin at week 5 to clear the infection (see e.g., FIG. 1A). The serum, bladders, and kidneys of treated mice, termed convalescent mice, were then analyzed for IgG specific to Abp1D_{RBD} and Abp2D_{RBD} to determine if an immune response was elicited against these adhesins. While two individuals developed a low level of Abp1D or Abp2D-specific IgG (AUC ~0.005), most animals did not generate any appreciable antigen-specific IgG response (AUC<0.002) (see e.g., FIG. 1B). The low levels of antigen specific IgG to the Abp1 and 2 adhesins was not surprising, as this has been observed in other pilus systems. Each pilus is comprised of thousands of rod subunits tipped by a single adhesin. When mice are infected with whole bacterial cells or immunized with whole pili, the antibody response is skewed towards the much more abundant rod subunit and the response to the adhesin is minimal. That said, CAUTI mice treated with antibiotics and subsequently re-infected displayed a reduction in bladder and catheter bacterial titers of approximately 1 log compared to naive mice (see e.g., FIG. 1C). The lack of IgG titers suggests that factors other than adhesin-specific IgG, such as epithelial trained immunity or adaptive immunity to other bacterial epitopes, are likely responsible for the protective effect.

Immunization with A. Baumannii Abp1/Abp2 Adhesins Provides Protection from CAUTI.

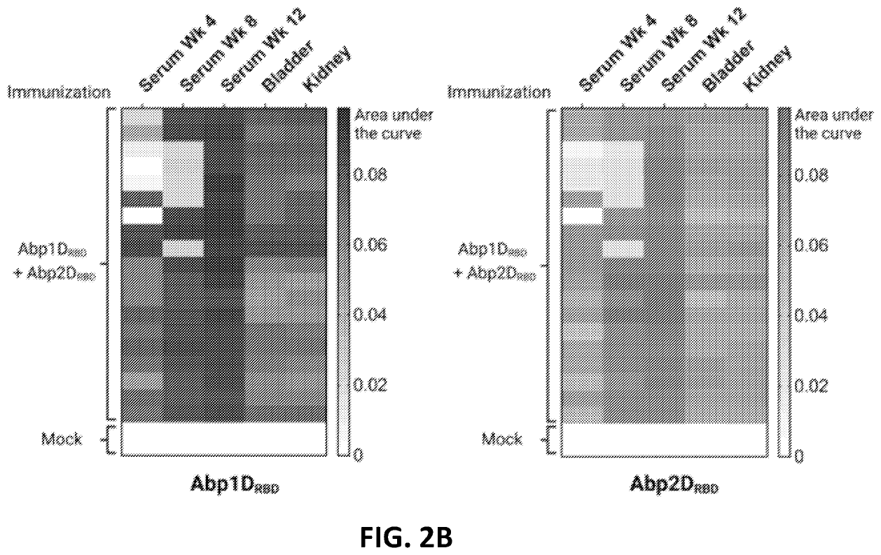

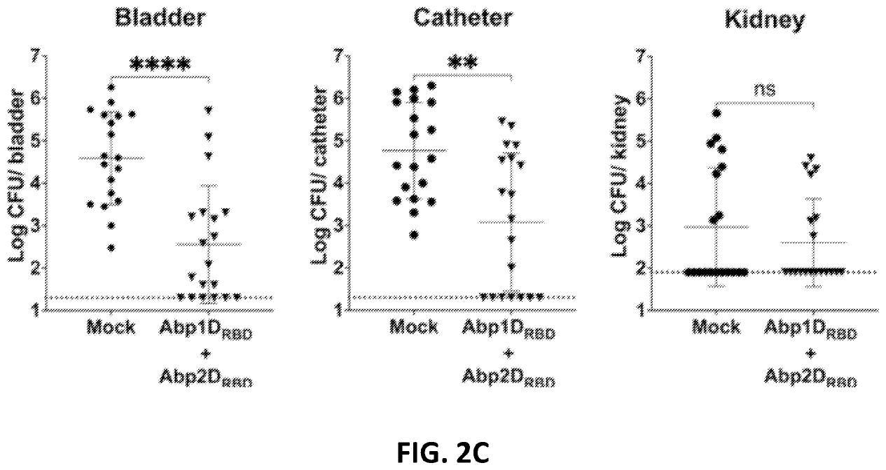

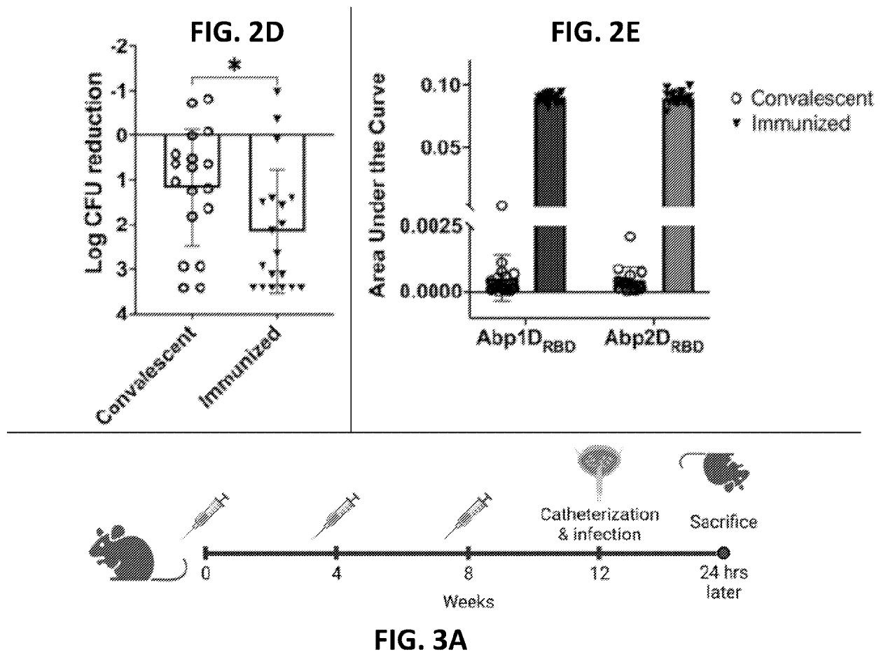

Mutations in abp1/abp2 attenuate virulence. Thus, based on work with other adhesin-based vaccines, it was hypothesized that immunization with the A. baumannii adhesins, Abp1D and Abp2D, might confer increased protection relative to natural immunity. The Abp1D and Abp2D receptor binding domains (RBDs) were purified as previously described (see e.g., Tamadonfar et al. (2023) Proc. Natl. Acad. Sci. U.S.A. 120, e2212694120) and the proteins used to immunize C57BL/6 mice. Four weeks after the final immunization, mice were catheterized and infected with strain ACICU (see e.g., FIG. 2A). Since the efficacy of most vaccines depends on eliciting a strong antigen-specific IgG response, serum was collected at: i) week 4 (prior to 2^{nd} immunization); ii) week 8 (prior to 3rd immunization); and iii) week 12 (at time of sacrifice), to test for Abp1 D_{RBD}- and Abp2D_{RBD}-specific IgG. Bladder and kidney homogenates collected at sacrifice were also tested for Abp1 D_{RBD}- and Abp2D_{RBD}-specific IgG. All animals produced a strong IgG response against both Abp1 D_{RBD} and Abp2D_{RBD}. This response was enhanced with each subsequent immunization to an AUC>0.08 at week 12 (see e.g., FIG. 2B). Mice that received Abp1D_{RBD}/Abp2D_{RBD} immunizations had significantly (1.5-2 log) lower bacterial titers in bladder tissue and on the catheter surface than mock immunized animals (see e.g., FIG. 2C). Remarkably, the magnitude of the phenotype was significantly increased compared to that observed in convalescent mice (see e.g., FIG. 2D). The degree of antigen-specific IgG in serum and bladder tissue was also much greater in immunized mice compared to convalescent mice (AUC>0.08 vs <0.005) (see e.g., FIG. 2E). These data demonstrate that Abp1D_{RBD}/Abp2D_{RBD} vaccination both produces greater immunity against two key CAUTI virulence factors than does natural infection and provides a superior level of protection.

Immunity to Abp2D_{RB}D, but not Abp1 D_{R}BD, is Required for Protection from A. Baumannii CAUTI.

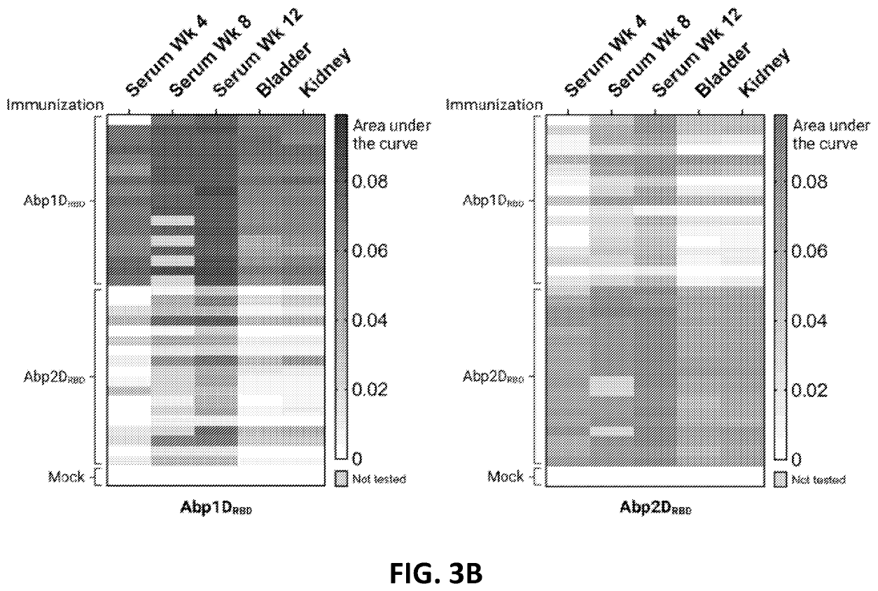

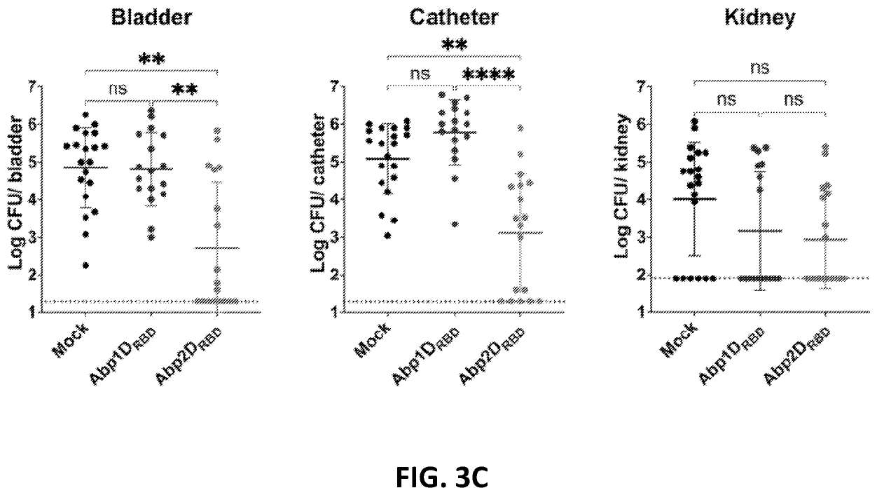

As mentioned above, A. baumannii deficient in either Abp1 D, Abp2D, or both are attenuated in a CAUTI model. To test whether immunity to both adhesins is required for protection from CAUTI, mice were immunized with each adhesin individually (see e.g., FIG. 3A). The cross-reactivity of the IgG response was tested in immunized animals (see e.g., FIG. 3B) because Abp1D_{RBD} and Abp2D_{RBD} share both structural homology and 70% sequence identity. Each mouse in the Abp1D_{RBD}-immunized group produced a strong Abp1D_{RBD}-specific IgG response, but the degree of cross-reactivity with Abp2D_{RBD} varied between animals, with only ~30% displaying strong cross-reactivity. Similarly, each mouse in the Abp2D_{RBD}-immunized group produced a strong Abp2D_{RBD}-specific IgG response, with strong Abp1 D_{RBD} cross-reactivity occurring in ~50% of individuals. Upon catheterization and infection, mice that received Abp2D_{RBD} immunizations were protected from infection, with a statistically significant 2-3 log decrease in bladder and catheter bacterial titers compared to mock-immunized animals (see e.g., FIG. 3C). However, despite high serum levels of Abp1 D_{RBD}-specific IgG (AUC>0.08), mice that received Abp1D_{RBD} immunizations were not protected from infection, with bladder and catheter titers equivalent to mock-immunized animals (see e.g., FIG. 3C).

This suggests that the lack of protection from CAUTI in Abp1 D_{RBD}-immunized animals is not due to a lack of immunogenicity. This study indicates that immunity to Abp2D_{RBD} alone is both necessary and sufficient for protection from A. baumannii CAUTI in this model.

### Abp2D_{RBD} Vaccine Generates Antigen-Specific Bone Marrow Plasma Cells and Splenic Memory B Cells.

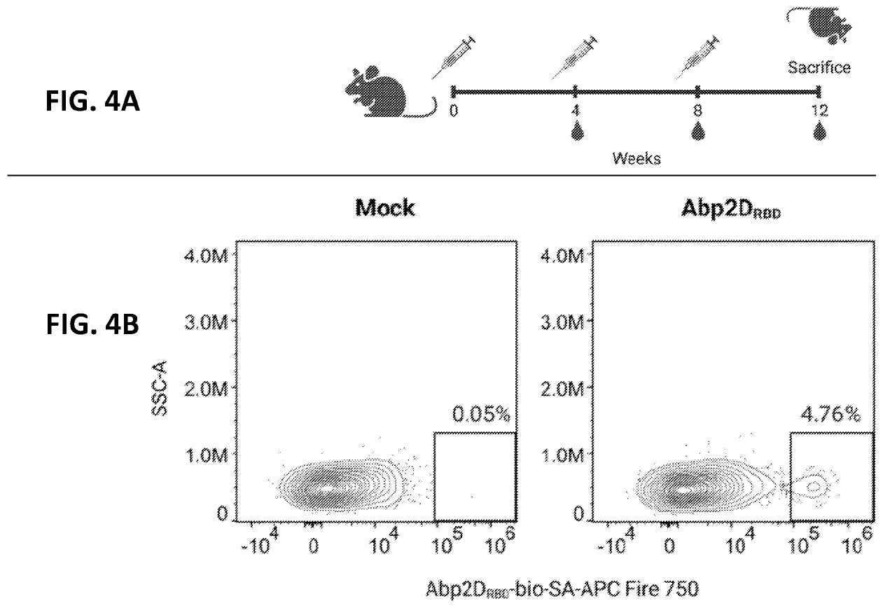

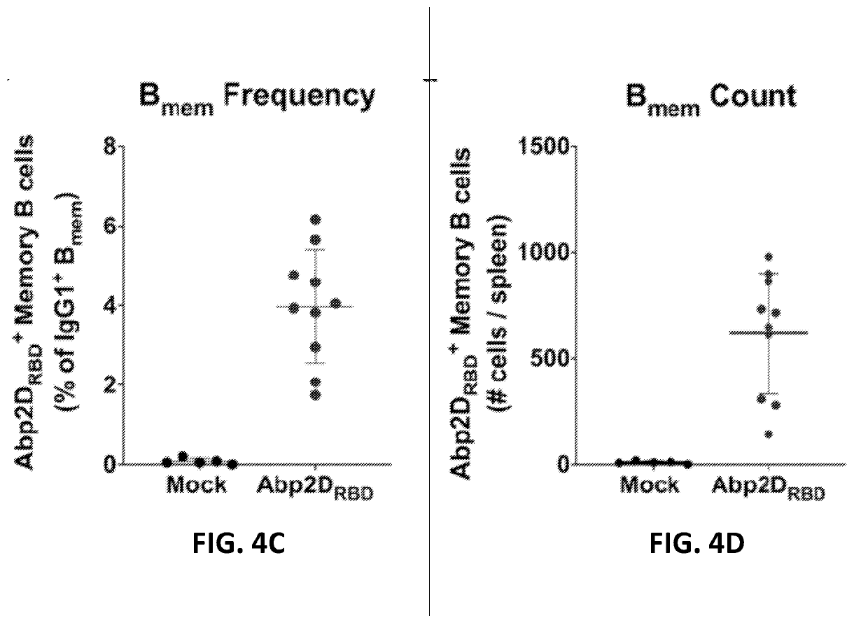

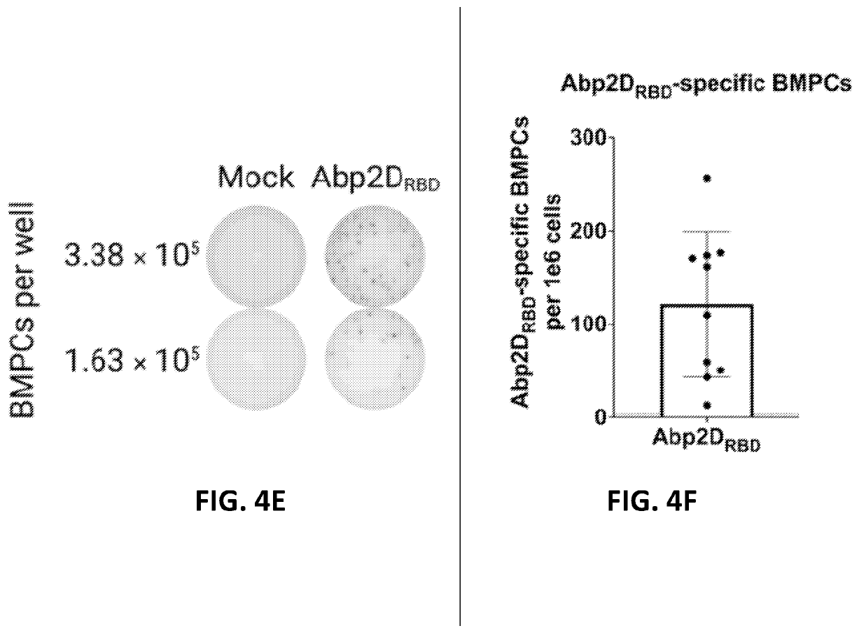

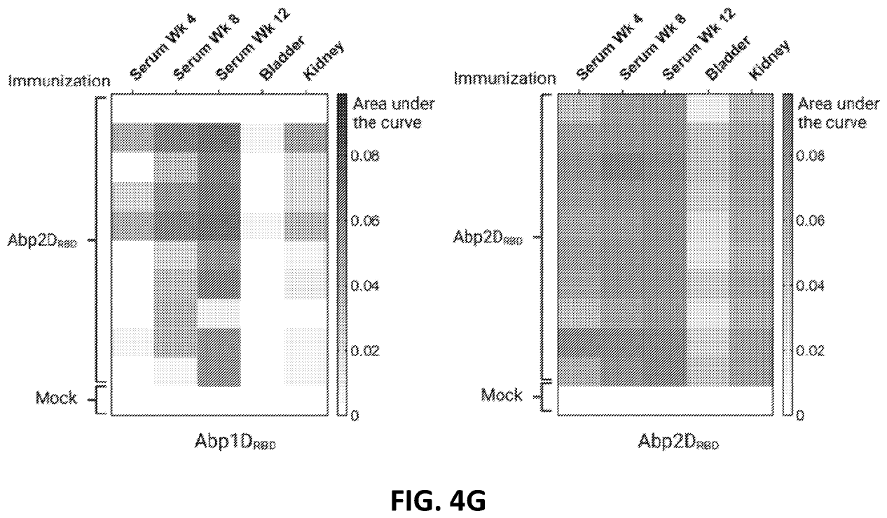

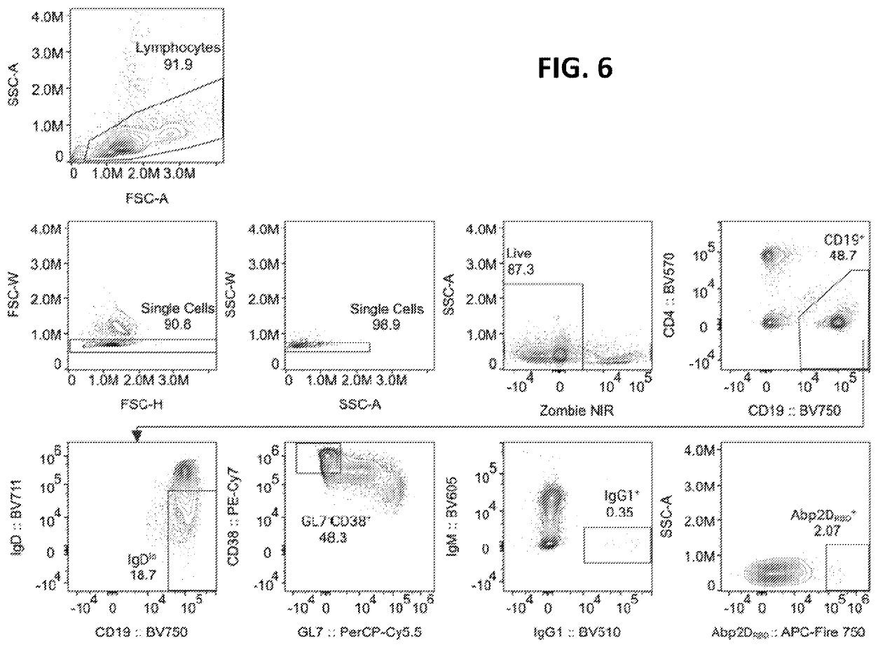

A successful vaccine elicits an antibody response that is both high-affinity and long-lasting. The immunogenicity of the Abp2D_{RB}D protein subunit vaccine was evaluated by examining memory B cells and bone marrow plasma cells in immunized animals (see e.g., FIG. 4A). Abp2D_{RBD}-specific memory B cells were detectable in the spleens of all immunized animals (see e.g., FIG. 4B-FIG. 4D) by flow cytometry. Antigen-specific memory B cells were defined as lymphocytes/single cells/live/CD4− CD19+/lgDlo/GL7−CD38+/IgG1+/Abp2D_{RBD}-bio-SA-APC-Fire750+(see e.g., FIG. 6). In addition, all immunized animals had detectable Abp2D_{RBD}-specific antibody-secreting cells in their bone marrow as assayed by ELISpot (see e.g., FIG. 4E and FIG. 4F). Serum and tissue IgG levels were tested in these mice, which were Abp2D_{RBD}-vaccinated but did not undergo CAUTI (see e.g., FIG. 4G). Serum and kidney titers were similar to those seen in earlier cohorts. However, bladder IgG levels were reduced (AUC≤0.04), likely due to a lack of catheterization, which is known to induce significant inflammation and IgG influx. The presence of antigen-specific bone marrow plasma cells, memory B cells, and high levels of serum IgG indicate that Abp2D_{RBD} vaccination generates all of the hallmarks of immunity of an effective vaccine.

Passive Immunization with Serum from Abp2D_{RBD}-Immunized Mice Protects Naïve Mice from CAUTI.

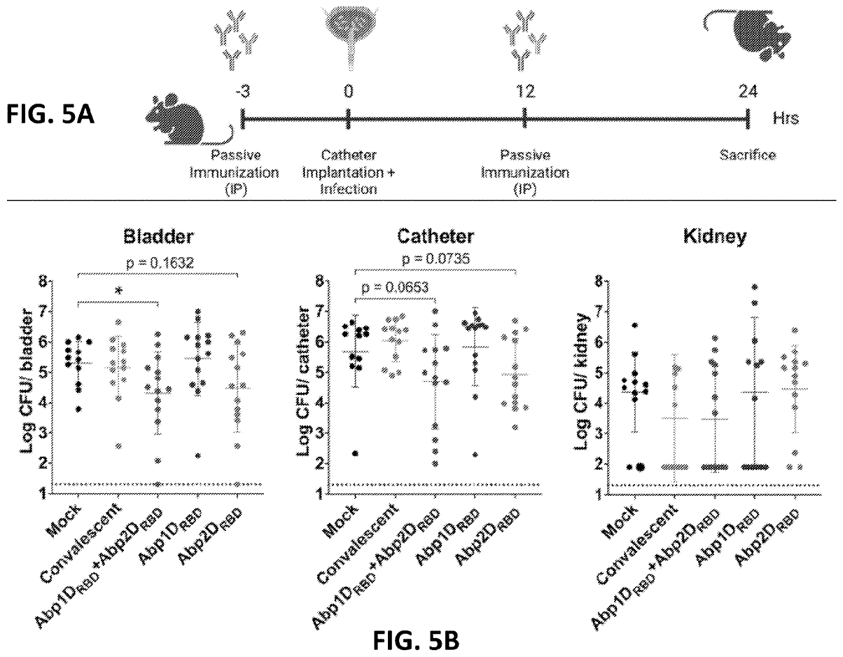

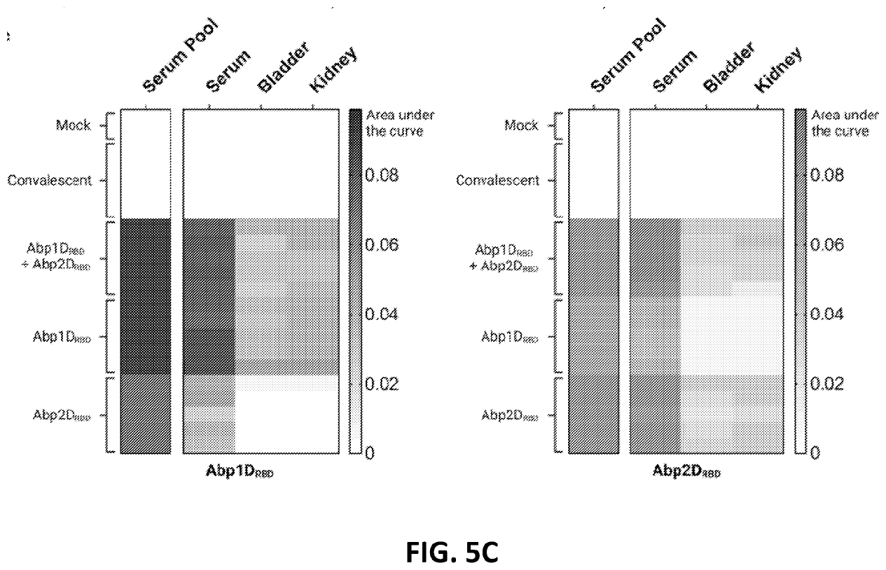

If the immunity conferred by the Abp2D_{RBD} vaccine is due to serum IgG, then transferring IgG from immunized animals to naive animals should also provide protection from a CAUTI challenge. To test the degree to which immunity conferred by the Abp2D_{RBD} vaccine is antibody-mediated, serum pooled from 5 groups of mice was administered: i) mock immunized; ii) convalescent; iii) Abp1D_{RBD}+Abp2D_{RBD} immunized; iv) Abp1D_{RBD}-immunized; and v) Abp2D_{RBD}-immunized (see e.g., FIG. 5A). Mice that received serum from Abp1D_{RBD}+Abp2D_{RBD} immunized animals had a statistically significant reduction of ~1 log in bladder bacterial titers (P<0.05), while mice receiving serum from convalescent or Abp1D_{RBD}-immunized mice were not protected (see e.g., FIG. 5B). Mice that received serum from Abp2D_{RBD}-immunized animals had a 1 log reduction in bladder bacterial titers trending towards significance (P=0.1632). The smaller effect size compared to vaccination is not unexpected, since passively immunized mice have a much lower concentration of adhesin-specific antibodies in their bladder and kidney tissues (see e.g., FIG. 5C). These data demonstrate that humoral rather than cellular immunity is the likely driver of protection in the vaccine model.

### Discussion

Catheter-associated urinary tract infections are the second most common cause of healthcare-associated infections. Although A. baumannii causes a small percentage of all CAUTIs, these infections are often multi-drug resistant and frequently life-threatening for affected patients, leading the CDC to label A. baumannii as a “pathogen of urgent concern.” Thus, there is a critical need to develop novel antimicrobial strategies to combat this infection. Herein is demonstrated that a vaccine targeting the interaction between A. baumannii and its ligand provides protection from CAUTI. The Abp2D_{RBD} vaccine described herein elicits many features of a successful immune response including memory B cells, bone marrow plasma cells, and high levels of serum and tissue IgG. While several vaccine strategies have been attempted for A. baumannii with mixed results, herein is believed to be the first report of an A. baumannii vaccine that is effective in preventing CAUTI pathogenesis in a mouse model.

Other adhesin-based vaccines for the treatment and prevention of urinary tract infections have been reported in the literature. A vaccine against the E. coli FimH pilus adhesin, which has been shown to be critical in interactions initiating and perpetuating E. coli cystitis, has recently completed a Phase Ia/Ib human clinical trial. This trial showed that the vaccine reduced the incidence of recurrent UTI by more than 75% in vaccinated patients. Analogous to COVID vaccines that target the SPIKE protein, the strategy described herein is to neutralize the adhesin that Acinetobacter uses for binding fibrinogen-coated catheters, leading to infection. Both Abp1 and Abp2 pili are capable of binding to fibrinogen, and both pili play a role in CAUTI. Thus, it was initially expected that immunity to both Abp1 D and Abp2D would be required in order to fully “neutralize” the bacteria and prevent adhesion to the catheter. It was therefore surprising that immunity to Abp1D_{RBD} proved to be unnecessary for protection from CAUTI. Although mice immunized with Abp1 D_{RBD} generated high serum levels of Abp1 D_{RBD}-specific IgG, including IgG capable of cross-reacting with Abp2D_{RBD}, there was no protective effect.

Structural studies of Abp1D_{RBD} and Abp2D_{RBD} provide a possible explanation for this observation. While the two adhesins share a great deal of structural similarly, the anterior loop of the binding pocket is considerably more flexible in Abp1 D_{RBD} than in Abp2D_{RBD}. Because of this flexibility Abp1 D_{RBD} can adopt either a “closed” (lower affinity) or “open” (higher affinity) conformation. An antibody response generated against the “closed” conformation is unlikely to recognize the binding pocket and therefore unlikely to functionally inhibit binding to a fibrinogen coated catheter. Conversely, the anterior loop of Abp2D_{RBD} is more rigidly positioned in an open conformation and therefore presents a more reliably accessible antibody epitope. The differences in both conformation and flexibility between the binding pockets of the two proteins may explain why Abp2D_{RBD} immunization is more protective than immunization with Abp1 D_{RBD}. Other explanations for the difference between Abp1 D_{RBD} and Abp2D_{RBD} vaccines may include changes in pilus expression, variations in epitope availability, or other differential factors.

Demonstrated herein is that the Abp2D_{RBD} vaccine produces a robust antigen-specific IgG response and that this immunity is transferable through serum. It remains to be understood which specific properties of the antibody response are providing the protection from challenge. It was initially hypothesized that protective antibodies would “neutralize” bacteria by physically blocking the interaction between Abp2D and its ligand, fibrinogen, to reduce catheter bacterial colonization and thus prevent bladder infection. Vaccinated mice demonstrate a reduction in catheter bacterial titers, including several individual animals that completely excluded A. baumannii colonization of the catheter, so this mechanism of action is plausible. However, antibodies may also promote infection clearance through other mechanisms such as opsonization and complement activation. Future studies will attempt to establish which properties of the Abp2D_{RBD} antibody response are most essential for protection and optimize Abp2D_{RBD} immunizations to maximize efficacy.

Vaccines have an important role to play in reducing the incidence of disease and decreasing opportunities for natural selection of antibiotic-resistance. However, it is difficult to predict which patients may develop an A. baumannii infection and therefore challenging to identify who would most benefit from vaccination. One potential patient cohort is chronically catheterized patients. CAUTI risk increases by 3-7% for each day of catheterization, leading to an almost 100% probability of CAUTI in patients who remain catheterized over the long term. Once established, CAUTI can be highly recurrent in spite of repeated antibiotic administration. Thus, chronically catheterized patients may be good candidates for prophylactic vaccination against CAUTI pathogens such as A. baumannii. In addition, given that A. baumannii can establish intracellular reservoirs within bladder epithelial cells, patients with a history of A. baumannii cystitis may benefit from vaccination to prevent recurrence. However, perhaps the greatest potential benefit of an A. baumannii vaccine lies in the developing world. The highest relative burden of deaths associated with antibiotic-resistant A. baumannii occurs in low and middle income countries. Health centers in Somalia and Kuwait report that A. baumannii accounts for up to 25% of CAUTIs in their facilities. In this setting, the storage conditions required for a protein subunit vaccine (e.g., simple refrigeration) present an advantage over more modern vaccine modalities.

The findings described herein highlight how basic research into microbial pathogenesis, such as the identification of pili implicated in CAUTI, can be translated into effective, antibiotic-sparing therapeutics. An Abp2D_{RBD} vaccine has the potential to reduce A. baumannii CAUTI incidence in vulnerable patient populations and has an important role to play in the fight against antimicrobial resistant infections.

### Methods

### General Bacteriology

Bacterial stocks were maintained as glycerol stocks at −80° C. Strains were streaked on LB-agar plates and incubated at 37° C. for 14-18 hours, at which time colonies were selected and used to inoculate liquid low-salt LB media (10 g tryptone, 5 g NaCl, and 5 g yeast extract per L). All bacterial cultures used in this study were grown statically at 37° C. for 24 hours followed by 1:1000 dilution and subculture for an additional 18-20 hours. Bacteria were spun down at 3000×g, washed 1× in PBS, resuspended at the specified OD600, and kept on ice until use. A. baumannii strain ACICU, representative of global clone 2, was used for all experiments described in this study (see e.g., Hamidian et al. (2019) Microb. Genomics 5, e000298).

### Protein Purification and Labeling

Protein was expressed and labeled as previously described (see e.g., Flores-Mireles et al. (2016) mBio 7). Briefly, cells were harvested in a large-scale fermenter format from C600 containing expression plasmids, grown to mid-logarithmic phase, and induced with 0.1 mM IPTG for 1 h. The culture was subsequently harvested, and the periplasm isolated generally as described previously (see e.g., Eldridge et al. (2020) Pathogens 9(1066)). RBD protein constructs and mutants were purified by cobalt affinity chromatography, eluted at ~150 mM imidazole with a gradient of 1×PBS to 1×PBS/300 mM imidazole. Protein-containing fractions were pooled and run on a Source 15S (Tₘ GE) cation-exchange column and eluted at 30 mM NaCl with a gradient of 20 mM MES pH 5.7 to 20 mM MES pH 5.7/200 nM NaCl. Purified protein was subsequently dialyzed or buffer exchanged into 20 mM MES pH 5.8+50 mM NaCl. Where required, protein was biotinylated using the EZ-Link NHS-PEG4 biotinylation reagent (Thermo Scientific) and diluted in H₂O to 100 mM. Protein was either dialyzed or buffer exchanged into 1×PBS. Biotinylation reagent was added at a 20 fold molar excess for 2 h at 4° C. under rocking. Biotinylated protein was subsequently dialyzed into PBS, removing the excess biotin reagent.

### Murine Immunizations

All immunizations were prepared by mixing 50 μg/mouse of Abp1D_{RBD} or Abp2D_{RBD} 1:1 by volume with Addavax, a squalene oil-in-water adjuvant (Invivogen) to a total volume of 50 μL/mouse. Mock immunizations were prepared by mixing buffer 1:1 with Addavax. C57Bl/6 mice were obtained from Charles River Laboratories and were 7-9 weeks old at the first immunization. Mice were immunized intramuscularly in the hind flank at weeks 0, 4, and 8, for a total of 3 immunizations of 50 μg/protein each. For dual immunization experiments, each mouse received 50 μg of Abp1D_{RBD} in the left hind flank and 50 μg of Abp2D_{RBD} in the right hind flank at each time point. Blood was collected at weeks 4 and 8 by submandibular or submental collection prior to the administration of the immunization.

### Murine CAUTI Model

Mice were catheterized and infected as described previously (see e.g., Di Venanzio et al. (2019) Nat. Commun. 10(2763). Briefly, mice were anesthetized with 4% isoflurane/0.8% oxygen by inhalation. A short piece of silicone tubing (4-5 mm) was transurethrally inserted into the bladder and immediately followed by 2 doses of 2×10⁸ CFUs of A. baumannii strain ACICU in 50 μl of PBS (OD600~13). 24 hours after infection, mice were anaesthetized and humanely sacrificed by cervical dislocation. Blood was collected from the inferior vena cava, allowed to clot for 30 minutes at room temperature, and spun down to remove red blood cells and clotting factors. Serum was removed to a new tube and frozen at −20° C. until analysis. Catheters were removed from bladders, placed into 1 mL of sterile PBS, and processed by vortexing for 30 seconds, sonicating for 10 minutes, and vortexing for an additional 30 seconds to remove biofilm and bacteria from the catheter surface. Bladders and kidney pairs were both placed into tubes containing sterile stainless steel beads and sterile PBS (1 ml for bladders, 800 μl for kidney pairs) and homogenized at 4° C. using the MP Biomedical Fastprep-24 homogenizer. The homogenization settings used were 1 min shaking at 4 m/s, 5 min of rest, followed by an additional 1 min of shaking. Bladder, kidney, and catheter samples were serially diluted and plated on selective media (LB+100 μg/L Ampicillin). Plates were incubated at 37° C. for 12-16 hours and bacterial cfus enumerated. Remaining bladder and kidney homogenates were frozen at −20° C. for additional analyses.

### Convalescent Infection Model

7-9 week old C57Bl/6 mice were catheterized and infected as described above. One group of mice received 2 doses of 2×10⁸ cfus of ACICU, and the other group received sterile PBS. Urines were collected at days 3, 7, 10, 14, and weekly thereafter. Blood was collected by submandibular or submental collection at weeks 3, 5, and 8, and at time of sacrifice. At week 5, mice were treated with 1 g/L Apramycin for 10 days to clear bacteriuria. At week 8, mice were again catheterized and infected, then sacrificed at 24 hours post infection as described above.

### ELISAs

All ELISAs were performed using Grenier Microlon high-binding plates (Grenier Bio-One #655085). Plates were coated with 100 μl of 1 μg/ml Abp1 D_{RBD}, Abp2D_{RBD}, or E. faecalis EbpANTD (used as a negative control for anti-HIS antibodies) in PBS and incubated overnight at 4° C. The following morning plates were washed 1× with 200 μl PBS containing 0.05% Tween-20 (PBS-T). Plates were blocked with 300 μl of PBS-T containing 10% fetal bovine serum (P10) for 1.5 hours at room temperature. Serum, bladder, and kidney homogenates were diluted 1:30 into 75 μl P10 and then serially diluted 1:3 and incubated for 1 hour at room temperature. Plates were washed 3× in PBS-T. Goat anti-mouse-IgG-HRP secondary antibody (Southern Biotech Cat #1030-05) was diluted 1:1000 in P10 and 100 μl added to each well and incubated for 1 hour at room temperature in the dark. Plates were washed 3× with PBS-T followed by 3× with PBS, developed with 100 μl developing reagent and quenched with 100 μl of 1 M HCl. Developing reagent consists of 10 ml phosphate-citrate buffer (Sigma Cat #P4809), 4 mg o-Phenylenediamine dihydrochloride (Sigma Cat #P8787), and 33 μl 3% H₂O₂ per plate. Plates were read using the BioTek ELx800 plate reader on the OD490 setting. Graphpad Prism 9 was used to calculate area under the curve for each sample. AUCs were baseline corrected by subtracting the AUC binding to the negative control protein, EbpANTD,23 which contains the same 6× His tag used 367 to purify Abp1 D_{RBD} and Abp2D_{RBD} but is otherwise structurally unique.

### ELISpot

PVDF-membrane plates (Millipore Sigma #MSIPN4W50) were prepared by activating with 50 μl of 35% ethanol for 30 seconds followed by washing 3× with PBS. Plates were coated with 100 μl of 5 μg/ml Abp2D_{RBD} or anti-mouse IgG (positive control) in PBS and incubated overnight at 4° C. The next morning, plates were washed 3× with PBD+0.05% Tween-20 (PBS-T) and blocked with 200 μl of RPMI media containing 10% fetal bovine serum (R10) for 2 hours at 37° C. and 5% C02. Mice that were immunized as described above were sacrificed 4 weeks after the third immunization. Bone marrow was collected from both femurs into R10, washed, and resuspended to a concentration of 1×10⁷ cells/ml. 5×10⁵ cells were added to the first well and serially diluted. Plates were incubated for 4 hours at 37° C. and 5% C02. Plates were washed 1× with PBS and 3× with PBS-T. 100 μl of biotinylated anti-mouse IgG (Southern Biotech Cat #1030-08) diluted 1:1000 in PBS containing 2% fetal bovine serum and 2 mM EDTA was added to the plate and incubated overnight at 4° C. The next day, plates were washed 3× with PBS-T. HRP-conjugated streptavidin (Jackson Immunoresearch Cat #016-030-084) was diluted 1:5000 in PBS+2% FBS/2 mM EDTA, 100 μl added to each well, and the plates incubated for 1.5 hours at room temperature in the dark. Plates were washed 3× with PBS-T followed by 1× with PBS. Developing solution was prepared by diluting 3 mg of 3-amino-9-ethylcarbazole in 10 ml of 0.1 M sodium acetate buffer, pH 5.0 and syringe filtering through a 0.45 PVDF membrane. Just prior to use, 100 μl of 3% H₂O₂ was added to the mixture. 100 μl of developing solution was added to each well and allowed to incubate until spots were visible, ~5 minutes. Developing solution was removed and plates washed under DI water to halt the reaction. Plates were dried overnight at room temperature and imaged using the CTL ImmunoSpot imager (Cellular Technology Limited). Spots were counted using the CTL ImmunoSpot automatic counting program with default parameters.

### Flow Cytometry

Mice that were immunized as described above were sacrificed 4 weeks after the third immunization. Spleens were collected into RPMI containing 2% fetal bovine serum and manually homogenized using the back of a syringe plunger. Cells were filtered through 75 um mesh, washed 1×, and counted. 2×107 splenocytes were stained for flow cytometry. All washes for the staining process were performed in PBS containing 2% fetal bovine serum and 2 mM EDTA. Cells were incubated with CD16/32 (Biolegend Cat #101302) and 5.875 μg/ml of biotinylated Abp2D_{RBD} for 10 minutes, then washed 3×. A cocktail containing the following antibodies was prepared in BD Brilliant Staining Buffer (BD Cat. #563794), all sourced from BioLegend unless otherwise indicated: Zombie NIR (Cat #423105), CD19-BV750 (Cat #115561), CD4-BV570 (Cat #100542), IgD-BV711 (Cat #405731), IgM-BV605 (Cat #406523), IgG1-BV510 (Cat #406621), Fas-PE (BD Cat #554258), GL7-PcpCy5.5 (Cat #144610), CD38-PE-Cy7 (Cat #102718), CD138-BV421 (Cat #142508), and streptavidin-APC-Fire-750 (Cat #405250). Invitrogen UltraComp eBeads were used for single colors. Flow cytometry data was collected using the Cytek Aurora with 4 laser 16V-14B-10YG-8R configuration and processed on FlowJo10 for Mac.

### Passive Immunization Model

Serum was collected at the time of sacrifice for all immunized and convalescent animals described above, and this serum was used for passive immunization experiments. 200 μl of serum from each individual within a group was combined to form the serum pools. Serum pools were sterile filtered and frozen in aliquots at −20° C. until use. Five pools were prepared: i) Mock immunized/Mock infected, ii) Convalescent, iii) Abp1D_{RBD}+Abp2D_{RBD} immunized, iv) Abp1D_{RBD} immunized, v) Abp2D_{RBD} immunized. Naive, 7-9 week old C56Bl/6 mice received 1 dose of 100 μl pooled serum 3 hours prior to catheterization and infection as described above. Mice received a second dose of 100 μl pooled serum 12 hours post infection. Mice were sacrificed at 24 hpi and tissue titers enumerated as described above.

### Statistical Analysis

All statistical tests were performed using built-in statistical functions of GraphPad Prism 9. All data analyzed for statistical significance (e.g., bacterial titer data) were nonparametric. The Mann-Whitney U test was used for comparisons of 2 groups. The Kruskal-Wallis test with multiple comparisons correction was used for comparisons of 3 or more groups.

What is claimed is:

1. A vaccine comprising:
at least one isolated Acinetobacter chaperone-usher pathway (CUP) adhesin protein or immunogenic fragment thereof; and
a pharmaceutically acceptable carrier or adjuvant.

2. The vaccine of claim 1, wherein the at least one isolated Acinetobacter CUP adhesin protein or immunogenic fragment thereof is derived from Acinetobacter baumannii.

3. The vaccine of claim 1, wherein the at least one isolated Acinetobacter CUP adhesin protein or immunogenic fragment thereof is Abp2D, Abp1 D, or a combination thereof.

4. The vaccine of claim 3,
wherein the at least one isolated Acinetobacter CUP adhesin protein or immunogenic fragment thereof is Abp2D; and
wherein the vaccine does not comprise Abp1 D.

5. The vaccine of claim 1, wherein the at least one isolated Acinetobacter CUP adhesin protein or immunogenic fragment thereof is selected from the group consisting of a receptor binding domain (RBD) of Abp2D, an RBD of Abp1 D, and a combination thereof.

6. The vaccine of claim 5,
wherein the at least one isolated Acinetobacter CUP adhesin protein or immunogenic fragment thereof is an RBD of Abp2D; and
wherein the vaccine does not comprise an RBD of Abp1 D.

7. The vaccine of claim 1, wherein the at least one isolated Acinetobacter CUP adhesin protein or immunogenic fragment thereof comprises a polypeptide sequence selected from the group consisting of SEQ ID NO: 1, SEQ ID NO: 2, SEQ ID NO: 3, SEQ ID NO: 5, SEQ ID NO: 6, SEQ ID NO: 7, and combinations thereof.

8. The vaccine of claim 1, wherein the at least one isolated Acinetobacter CUP adhesin protein or immunogenic fragment thereof comprises a polypeptide sequence having at least about 70% identity to SEQ ID NO: 1, SEQ ID NO: 2, SEQ ID NO: 3, SEQ ID NO: 5, SEQ ID NO: 6, or SEQ ID NO: 7.

9. The vaccine of claim 1, wherein the pharmaceutically acceptable carrier or adjuvant is selected from the group consisting of a cream, emulsion, gel, liposome, nanoparticle, or ointment.

10. The vaccine of claim 9, wherein the pharmaceutically acceptable carrier or adjuvant is squalene.

11. A method of treating or preventing a urinary tract infection (UTI) in a subject in need thereof, the method comprising administering to the subject a vaccine comprising at least one isolated Acinetobacter chaperone-usher pathway (CUP) adhesin protein or immunogenic fragment thereof and a pharmaceutically acceptable carrier or adjuvant.

12. The method of claim 11, wherein the UTI is a catheter-associated UTI (CAUTI).

13. The method of claim 12, wherein the subject is a chronically catheterized subject or has recurrent CAUTI.

14. The method of claim 11, wherein the at least one isolated Acinetobacter CUP adhesin protein or immunogenic fragment thereof is derived from Acinetobacter baumannii.

15. The method of claim 11, wherein the at least one isolated Acinetobacter CUP adhesin protein or immunogenic fragment thereof is Abp2D, Abp1 D, or a combination thereof.

16. The method of claim 15,
wherein the at least one isolated Acinetobacter CUP adhesin protein or immunogenic fragment thereof is Abp2D; and
wherein the vaccine does not comprise Abp1 D.

17. The method of claim 11, wherein the at least one isolated Acinetobacter CUP adhesin protein or immunogenic fragment thereof is selected from the group consisting of a receptor binding domain (RBD) of Abp2D, an RBD of Abp1 D, and a combination thereof.

18. The method of claim 17,
wherein the at least one isolated Acinetobacter CUP adhesin protein or immunogenic fragment thereof is an RBD of Abp2D; and
wherein the vaccine does not comprise an RBD of Abp1 D.

19. The method of claim 11, wherein the at least one isolated Acinetobacter CUP adhesin protein or immunogenic fragment thereof comprises a polypeptide sequence selected from the group consisting of SEQ ID NO: 1, SEQ ID NO: 2, SEQ ID NO: 3, SEQ ID NO: 5, SEQ ID NO: 6, SEQ ID NO: 7, and combinations thereof.

20. The method of claim 11, wherein the at least one isolated Acinetobacter CUP adhesin protein or immunogenic fragment thereof comprises a polypeptide sequence having at least about 70% identity to SEQ ID NO: 1, SEQ ID NO: 2, SEQ ID NO: 3, SEQ ID NO: 5, SEQ ID NO: 6, or SEQ ID NO: 7.
